(19) United States
(12) Patent Application Publication
(10) Pub. No.: US 2007/0032504 A1
(43) Pub. Date: Feb. 8, 2007

(54) Polymorphic form of 3-phenylsulfonyl -8-piperazin-1-yl-quinoline

(76) Inventor: Asa Elisabeth Gladwin, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/572,670

(22) PCT Filed: Sep. 23, 2004
(86) PCT No.: PCT/EP04/10843

§ 371 Date: Mar. 20, 2006

(30) Foreign Application Priority Data
Sep. 26, 2003 (GB) 0322629.7

(51) Int. Cl. A61K 31/496; C07D 403/02

(52) U.S. Cl. 514/253.07; 544/363

(57) ABSTRACT

This invention relates to a novel compound having pharmacological activity, to processes for its preparation, to compositions containing it and to its use in the treatment of CNS and other disorders.

This invention relates to a novel compound having pharmacological activity, to processes for its preparation, to compositions containing it and to its use in the treatment of CNS and other disorders.

WO 2003/080580 (Glaxo Group Limited) describes the preparation of 3-phenylsulfonyl-8-piperazin-1-yl-quinoline (Example 16) in addition to two polymorphic forms of 3-phenylsulfonyl-8-piperazin-1-yl-quinoline (Form I; Example 51 and Form II; Example 52). 3-Phenylsulfonyl-8-piperazin-1-yl-quinoline is disclosed in WO 2003/080580 as having affinity for the 5-HT₆ receptor and is claimed to be useful in the treatment of CNS and other disorders.

It has now been found that 3-phenylsulfonyl-8-piperazin-1-yl-quinoline exists in a further polymorphic form which is primarily characterised in that it possesses a higher melting point than Forms I and II. This further polymorphic form of 3-phenylsulfonyl-8-piperazin-1-yl-quinoline is referred to as Form III.

Thus, according to a first aspect of the present invention, we provide 3-phenylsulfonyl-8-piperazin-1-yl-quinoline Form III.

Suitably, the present invention provides 3-phenylsulfonyl-8-piperazin-1-yl-quinoline, suitably as characterised by data provided by at least one of the following: infrared, Raman, X-ray powder diffraction or nuclear magnetic resonance and melting point data as provided herein, including partial spectral data provided herein.

Thus, one aspect of the present invention provides a polymorphic form of 3-phenylsulfonyl-8-piperazin-1-yl-quinoline, characterised in that it provides:

(i) an infrared spectrum containing peaks at 724, 758, 777, 804, 818, 838, 856, 905, 918, 948, 1023, 1055, 1081, 1092, 1118, 1136, 1153, 1178, 1244, 1302, 1318, 1365, 1378, 1403, 1444, 1471, 1490, 1569, 1584, 1603 and 2819 cm⁻¹; and/or

(ii) a Raman spectrum containing peaks at 159, 184, 214, 241, 285, 304, 318, 429, 545, 558, 614, 706, 724, 803, 856, 1000, 1023, 1080, 1093, 1136, 1152, 1233, 1243, 1317, 1343, 1364, 1378, 1403, 1446, 1569, 1584, 1602, 3050 and 3073 cm⁻¹; and/or

(iii) an X-ray powder diffraction (XRPD) pattern which gives calculated lattice spacings at 10.29, 11.94, 17.47, 19.55, 19.84, and 20.33°; and/or

(iv) a melting point of 188° C.

In one preferred aspect, the polymorph of the present invention provides an infrared spectrum substantially in accordance with FIG. 1.

In one preferred aspect, the polymorph of the present invention provides a Raman spectrum substantially in accordance with FIG. 2.

In one preferred aspect, the polymorph of the present invention provides an X-ray powder diffraction (XRPD) pattern which gives calculated lattice spacings at 10.29, 10.76, 11.94, 14.33, 14.61, 14.93, 16.02, 16.80, 17.47, 17.92, 19.13, 19.55, 19.84, 20.33, 21.16, 21.36, 23.33, 23.96, 24.44, 24.67, 25.51, 26.12, 27.13, 27.77, 28.06, 28.35, 29.23, 29.46, 30.06, 30.35, 31.27, 32.35, 32.66, 33.08, 33.77, 34.49, 35.18, 36.42, 37.34, 38.39 and 39.51°.

In a more preferred aspect, the polymorph of the present invention provides an X-ray powder diffraction (XRPD) pattern substantially in accordance with FIG. 3.

The present invention encompasses the polymorph isolated in pure form or when admixed with other materials, for example the known forms of 3-phenylsulfonyl-8-piperazin-1-yl-quinoline or any other material. Thus, in one aspect there is provided the polymorph in isolated form. In a further aspect there is provided the polymorph in pure form. In yet a further aspect there is provided the polymorph in crystalline form. Most preferably, the polymorph is provided in pure form.

‘Isolated in pure form’ refers to 3-phenylsulfonyl-8-piperazin-1-yl-quinoline Form III present in an amount of preferably >75%, more preferably >90%, particularly >95%, especially>99% relative to other compounds or polymorphs of 3-phenylsulfonyl-8-piperarazin-1-yl-quinoline which may be present in an isolated sample.

The compound of the present invention has affinity for the 5-HT₆ receptor and is believed to be of potential use in the treatment of certain CNS disorders such as anxiety, depression, epilepsy, obsessive compulsive disorders, migraine, cognitive memory disorders (e.g. Alzheimers disease, age related cognitive decline and mild cognitive impairment), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury such as hydrocephalus. The compound of the invention is also expected to be of use in the treatment of certain GI (gastrointestinal) disorders such as IBS (Irritable Bowel Syndrome). The compound of the invention is also expected to be of use in the treatment of obesity.

Thus the invention also provides 3-phenylsulfonyl-8-piperazin-1-yl-quinoline (Form III), for use as a therapeutic substance, in particular in the treatment or prophylaxis of the above disorders. In particular the invention provides for 3-phenylsulfonyl-8-piperazin-1-yl-quinoline (Form III), for use in the treatment of depression, anxiety, Alzheimers disease, age related cognitive decline, ADHD, obesity, mild cognitive impairment, schizophrenia, cognitive deficits in schizophrenia and stroke.

The invention further provides a method of treatment or prophylaxis of the above disorders, in mammals including humans, which comprises administering to the sufferer a therapeutically effective amount of 3-phenylsulfonyl-8-piperazin-1-yl-quinoline (Form III).

In another aspect, the invention provides the use of 3-phenylsulfonyl-8-piperazin-1-yl-quinoline (Form III) in the manufacture of a medicament for use in the treatment or prophylaxis of the above disorders.

5-HT₆ antagonists have the potential to be capable of increasing basal and learning-induced polysialylated neuron cell frequency in brain regions such as the rat medial temporal lobe and associated hippocampus, as described in WO 2003/066056. Thus, according to a further aspect of the present invention, we provide a method of promoting neuronal growth within the central nervous system of a mammal which comprises the step of administering 3-phenylsulfonyl-8-piperazin-1-yl-quinoline (Form III).

In order to use the compound of the present invention in therapy, it will normally be formulated into a pharmaceutical composition in accordance with standard pharmaceutical practice. The present invention also provides a pharmaceutical composition, which comprises 3-phenylsulfonyl-8-piperazin-1-yl-quinoline (Form III), and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtrabon. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrates the preparation of the compound of the invention.

Description 1

### 3-Iodo-8-nitroquinoline (D1)

A stirred mixture of 8-nitroquinoline (100 g, 0.57 mol) in acetic acid (500 ml) was treated with N-iodosuccinimide (155 g, 0.69 mol) portionwise over 10 minutes, and warmed to 62° C. for 6 h. A further portion of N-iodosuccinimide (25 g, 0.14 mol) was introduced and the mixture stirred for a further 16 h before cooling to ambient temperature. The solvent was removed in vacuo, keeping the temperature below 35° C. The residue was dissolved in dichloromethane (2 L) and washed successively with saturated aqueous sodium bicarbonate solution (2×1 L), 10% aqueous sodium thiosulphate solution (1 L), water (1 L), brine (100 ml), then the organic phase was dried over magnesium sulphate. The mixture was filtered and the solvent removed to give a yellow solid which was recrystallised from ethyl acetate to give the title compound (D1) (168 g, 97%) as a yellow solid;

δ_{H} (CDCl₃) 7.65 (1H, app.t), 7.94 (1H, dd), 8.07 (1H, dd), 8.66 (1H, d, J=2 Hz), 9.19 (1H, d, J=2 Hz);

Mass Spectrum: C₉H₅IN₂ requires 300; found 301 (MH⁺).

Description 2

### 8-Nitro-3-phenylsulfonylquinoline (D2)

3-Iodo-8-nitroquinoline (D1) (135 g, 0.45 mol), was suspended in dimethylformamide (2.4 L) in a 5 L 3-necked flask fitted with an overhead stirrer, under an argon atmosphere. This mixture was treated successively with anhydrous sodium phenylsulfinate (99.6 g 0.608 mol), and bis-(copper (I) triflate) benzene complex (170 g, 0.338 mol). The resulting slurry was heated to 65° C. for 18 h. The mixture was cooled, filtered and the solvent evaporated in vacuo. Acetone (2.5 L) was added to the residue and the solution filtered. The filtrate was evaporated in vacuo, a further 2.5 L of acetone added and the mixture filtered again. The solvent was evaporated in vacuo and the residue dissolved in chloroform (3 L) and washed with 10% aqueous ammonia (2×2 L), and the organic phase was dried over magnesium sulphate and the solvent evaporated in vacuo. The dark brown residue was purified using a Biotage flash-150 chromatography apparatus (5 kg silica gel) eluting with hexane and increasing proportions of ethyl acetate to give the title compound (D2) (81.5 g, 58%) as a yellow solid;

δ_{H} (d6-DMSO) 7.67 (2H, t), 7.57 (1H, d, 7.96 (1H, t), 8.13 (2H, d), 8.51 (1H, d), 8.59 (1H, d), 9.42 (1H, d), 9.50 (1H, d);

Mass Spectrum: C₁₅H₁₀SO₄N₂ requires 314; found 315 (MH⁺).

Description 3

### 8-Amino-3-phenylsulfonylquinoline (D3)

A slurry of 8-nitro-3-phenylsulfonylquinoline (D2) (46.7 g, 172 mmol), in tetrahydrofuran (750 ml) was added to a stirred solution of 30% titanium (III) chloride in aqueous HCl (470 ml) [Supplied by BDH] cooled in an ice bath, at such a rate that the temperature was maintained below 35° C. Once the addition was completed, the solution was stirred for a further 10 minutes then water (1.5 L) was introduced and the mixture poured into a 5 L beaker. The rapidly stirred solution was treated by portionwise addition of solid potassium carbonate in order to attain pH ˜8.5. EDTA (250 g, 0.86 mol) was added and followed by further potassium carbonate to maintain pH ˜8.5. The mixture was extracted with dichloromethane (3×1 L) and the combined organic phase passed through a silica plug (500 g) eluting with further dichloromethane (1 L) and 10% ethyl acetate in dichloromethane (1 L). The combined organic phases were evaporated and the residue subjected to purification using Biotage Flash-75 chromatography apparatus (2 kg silica gel), eluting with dichloromethane and increasing proportions of ether to give the title compound (D3) (34.5 g, 72%) as a pale brown solid;

δ_{H} (CDCl₃) 5.0 (2H, br s), 7.02 (1H, dd), 7.25 (1H, dd), 7.44 (1H, t), 7.50-7.59 (3H, m), 8.00-8.40 (2H, m), 8.70 (1H, s), 0.09 (1H, s);

Mass Spectrum: C₁₅H₁₂SO₂N₂ requires 284; found 285 (MH⁺).

Description 4

### 8-Iodo-3-phenylsulfonylquinoline (D4)

8-Amino-3-phenylsulfonylquinoline (D3) (31.6 g, 0.11 mol) was dissolved in trifluoroacetic acid (60 ml) and the mixture evaporated. The resulting brown oil was dissolved in acetonitrile (200 ml) and added dropwise to a stirred solution of n-butyl nitrite (6.1 ml) in acetonitrile (300 ml) maintained at a temperature of <5° C. Once the addition was completed, the mixture was stirred for five minutes then tetra-(n-butyl)ammonium iodide (82 g, 0.22 mol) added portionwise, keeping the temperature below 10° C. The mixture was stirred for a further 20 minutes then concentrated in vacuo. The dark residue was subjected to flash-75 chromatography (2 kg silica gel), eluting with hexane and dichloromethane to give a brown solid. This was dissolved in dichloromethane (500 ml) and washed with 10% aqueous sodium thiosulphate (2×300 ml), dried over magnesium sulphate and concentrated to an orange solid. This was triturated with methanol to give the title compound (D4) (25.2 g, 75%) as a light yellow solid;

δ_{H} (CDCl₃) 7.39 (1H, t), 7.53-7.63 (3H, m), 7.96 (1H, d), 8.04 (2H, dd), 8.50 (1H, dd), 8.79 (1H, d), 9.32 (1H, d);

Mass Spectrum: C₁₅H₁₀NO₂SI requires 395; found 396 (MH⁺).

Description 5

### 3-Phenylsulfonyl-8-piperazin-1-yl-quinoline (D5)

A 100 ml three necked flask was charged with Pd₂(dba)₃ (174 mg, 0.19 mmol, 0.03 eq), 8-Iodo-3-phenylsulfonylquinoline (D4) (2.5 g, 6.33 mmol), 1,1′-bis-diphenylphosphenoferrocene (316 mg, 0.57 mmol), sodium tertbutoxide (851 mg, 8.86 mmol, 1.4 eq) and piperazine (2.72 g, 31.6 mmol, 5 eq). The flask was evacuated and filled with nitrogen 4 times then anhydrous 1,4-dioxane (17.5 ml, 7 vol) was added. The mixture was stirred and heated to 40° C. for 16½ hrs.

The dark solution was allowed to cool to room temperature, dichloromethane (12.5 ml) was added and the solution was washed with H₂O (12.5 ml). The aqueous wash was extracted with dichloromethane and the combined organic layers were extracted with 5M HCl (2×12.5 ml). The combined aqueous layers were washed with (dichloromethane 2.5 ml) then transferred to a conical flask, dichloromethane (12.5 ml) was added and the flask was cooled in an ice/water bath. 10M Aqueous sodium hydroxide (13 ml) was added whilst stirring, the mixture was then stirred at room temperature until all the solids were dissolved. The lower organic layer was removed and the aqueous layer was extracted with dichloromethane (7.5 ml), the combined organic layers were concentrated under reduce pressure to ˜5 ml. Isooctane (2.5 ml) was added to the dark brown solution resulting in crystallisation, the mixture was stirred at room temp for 5 min then isooctane (22.5 ml) was added over 5 min. The mixture was aged at room temp for 1½ hrs before being cooled in an ice/water bath for 30 min, the mixture was filtered and the cake washed with isooctane (5 ml). The cake was dried under reduced pressure to give the title compound D5; yield 1.67 g, 75%.

δ_{H} (CDCl₃): 1.6 (1H, bs), 3.18 (4H, m), 3.34 (4H, m), 7.27 (1H, m), 7.49-7.60 (5H, m), 8.01 (2H, dd), 8.75, (1H, d), 9.21 (1H, d).

### EXAMPLE 1

### Crystallisation of 3-phenylsulfonyl-8-piperazin-1-yl-quinoline (Form III)

3-Phenylsulfonyl-8-piperazin-1-yl-quinoline (D5) (5 g) was dissolved in isopropanol (60 ml) with warming to reflux. The resultant solution was then treated with charcoal (1.25 g), filtered, then the solution was allowed to cool to ambient, crystallisation initiating at −60° C. The product was collected by filtration, washed with isopropanol (10 ml;) and dried in vacuo at 45° C. to give the title compound, 3.1 g, 62%. Melting point 188° C.

### EXAMPLE 2

### Crystallisation of 3-phenylsulfonyl-8-piperazin-1-yl-quinoline (Form III)

(Alternative Procedure)

3-Phenylsulfonyl-8-piperazin-1-yl-quinoline (D5) (10 g) was dissolved in ethanol (80 ml) with warming to 70° C. The resultant solution was then treated with charcoal (1 g), filtered, and the filter bed rinsed with ethanol (20 ml). The combined filtrate was adjusted to 54° C., then cooled to 50° C. over 15 minutes. The solution was seeded with 3-phenylsulfonyl-8-piperazin-1-yl-quinoline (Form III) (10 mg), cooled to 35° C. over 30 minutes, held for 1 hour, then cooled to 20° C. over 30 minutes, and stirred for a further 1 hour 15 minutes. The product was collected by filtration, washed with cold ethanol, (2×10 ml;) and dried in vacuo at 40° C. to give the title compound, 6.64 g, 66.4%. Melting point 188° C.

### EXAMPLE 3

### Crystallisation of 3-phenylsulfonyl-8-piperazin-1-yl-quinoline (Form III)

(Alternative Procedure)

3-Phenylsulfonyl-8-piperazin-1-yl-quinoline (D5) (10 g) was dissolved in ethanol (80 ml) with warming to 72° C. The resultant solution was then pumped through a Cuno R55SP filter into a second vessel (pre-heated to 55° C.) via a sinter funnel. The original flask and the Cuno filter were washed with ethanol (20 ml). The combined filtrate was heated to 72° C. to redissolve the material, then adjusted to 50° C. The solution was seeded with 3-phenylsulfonyl-8-piperazin-1-yl-quinoline (Form III) (10 mg), cooled to 35° C. over 30 minutes, held for 1 hour, then cooled to 20° C. over 30 minutes, and stirred for a further 48 hours. The product was collected by filtration, washed with ethanol (20 ml) and dried in vacuo at 50° C. to give the title compound, 5.64 g, 56.4%. Melting point 188° C.

Characterising data recorded for Example 1:

The infrared spectrum of the solid product was recorded using a Nicolet Avatar 360 FT-IR spectrometer fitted with a universal ATR accessory. The FT-IR spectrum (FIG. 1) shows bands at: 724, 758, 777, 804, 818, 838, 856, 905, 918, 948, 1023, 1055, 1081, 1092, 1118, 1136, 1153, 1178, 1244, 1302, 1318, 1365, 1378, 1403, 1444, 1471, 1490, 1569, 1584, 1603 and 2819 cm⁻¹.

FT-Raman spectra of samples in glass tubes were acquired using a ThermNicolet 960 E.S.P. spectrometer. Excitation at 1064 nm was provided by a Nd:YVO₄ laser with a power of 400 mW at the sample position. 1200 scans were recorded at 4 cm⁻¹ resolution.

The FT-Raman spectrum (FIG. 2) shows bands at 159, 184, 214, 241, 285, 304, 318, 429, 545, 558, 614, 706,724,803, 856,1000, 1023, 1080, 1093, 1136, 1152, 1233, 1243, 1317, 1343, 1364, 1378, 1403, 1446, 1569, 1584, 1602, 3050 and 3073 cm⁻¹.

The X-Ray Powder Diffractogram pattern of the solid product (FIG. 3) was recorded using the following acquisition conditions: Unground material was packed into top-filled Si cups. Powder patterns were obtained using a Bruker D8 Advance X-Ray powder diffractometer configured with a Cu anode (40 kV, 40 mA), variable divergence slit, primary and secondary Soller slits, and a position sensitive detector. Data were acquired over the range 2-40 degrees 2-theta using a step size of 0.0145 degrees 2-theta (1 s per step). Samples were rotated during data collection. Characteristic 2θ XRPD angles are 10.29, 10.76, 11.94, 14.33, 14.61, 14.93, 16.02, 16.80, 17.47, 17.92, 19.13, 19.55, 19.84, 20.33, 21.16, 21.36, 23.33, 23.96, 24.44, 24.67, 25.51, 26.12, 27.13, 27.77, 28.06, 28.35, 29.23, 29.46, 30.06, 30.35, 31.27, 32.35, 32.66, 33.08, 33.77, 34.49, 35.18, 36.42, 37.34, 38.39 and 39.51°.

### BRIEF DESCRIPTION OF THE DRAWINGS

FIG. 1 shows the Infrared spectrum obtained for 3-phenylsulfonyl-8-piperazin-1-yl-quinoline (Form III).

FIG. 2 shows the Raman spectrum obtained for 3-phenylsulfonyl-8-piperazin-1-yl-quinoline (Form III).

FIG. 3 shows the X-Ray Powder Diffractogram obtained for 3-phenylsulfonyl-8-piperazin-1-yl-quinoline (Form III).

1. A polymorphic form of 3-phenylsulfonyl-8-piperazin-1-yl-quinoline characterised in that it provides:
(i) an infrared spectrum containing peaks at 724, 758, 777, 804, 818, 838, 856, 905, 918, 948, 1023, 1055, 1081, 1092, 1118, 1136, 1153, 1178, 1244, 1302, 1318, 1365, 1378, 1403, 1444, 1471, 1490, 1569, 1584, 1603 and 2819 cm⁻¹; and/or
(ii) a Raman spectrum containing peaks at 159, 184, 214, 241, 285, 304, 318, 429, 545, 558, 614, 706, 724, 803, 856, 1000, 1023, 1080, 1093, 1136, 1152, 1233, 1243, 1317, 1343, 1364, 1378, 1403, 1446, 1569, 1584, 1602, 3050 and 3073 cm⁻¹; and/or
(iii) an X-ray powder diffraction (XRPD) pattern which gives calculated lattice spacings at 10.29, 11.94, 17.47, 19.55, 19.84, and 20.33°; and/or
(iv) a melting point of 188° C.

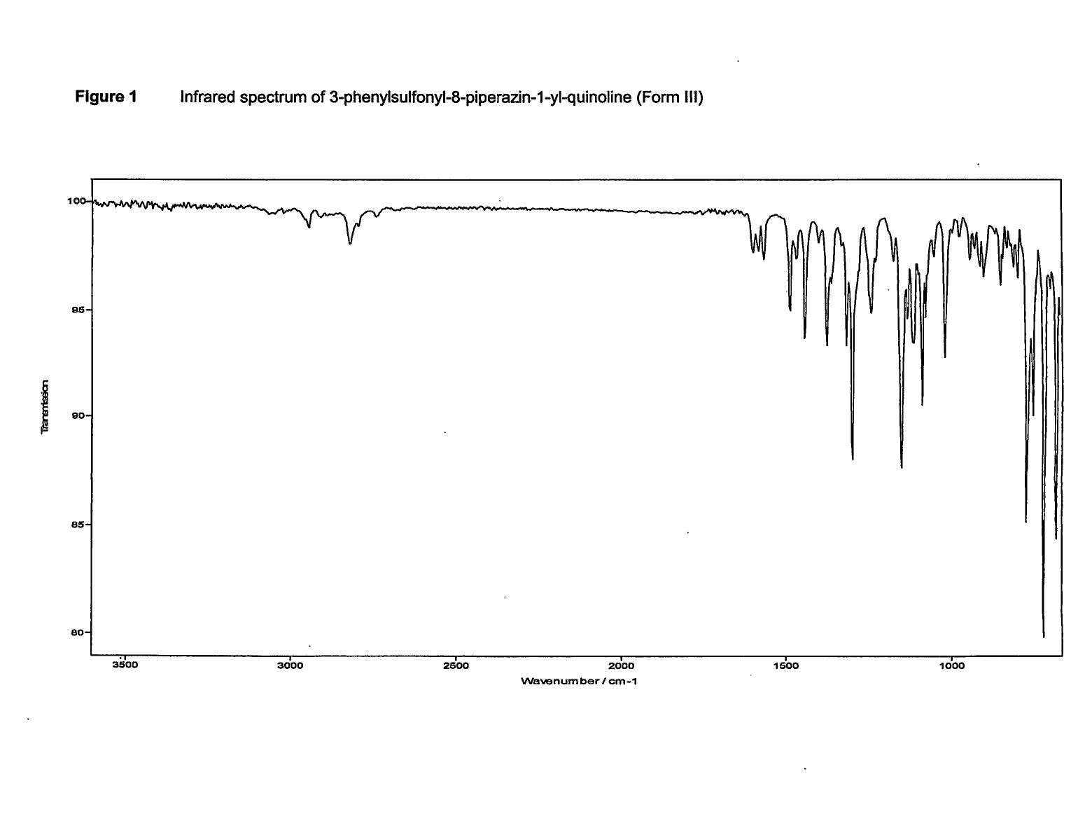

2. A polymorph according to claim 1 which provides an infrared spectrum substantially in accordance with FIG. 1.

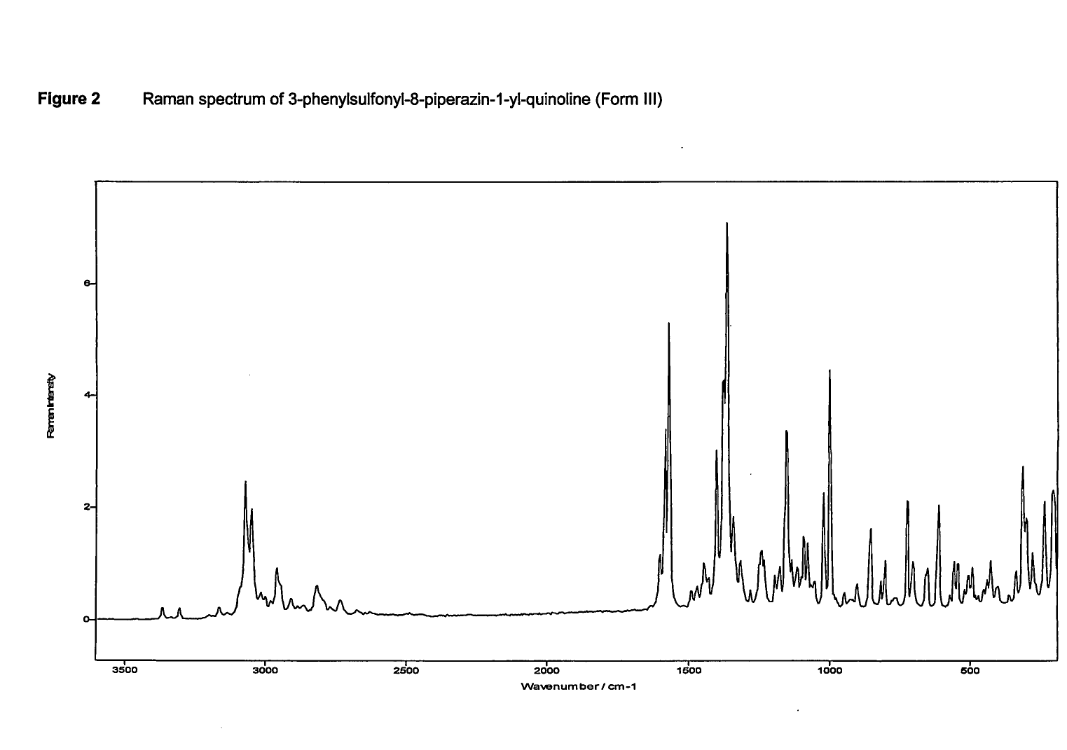

3. A polymorph according to claim 1 which provides a Raman spectrum substantially in accordance with FIG. 2.

4. A polymorph according to claim 1 which provides an X-ray powder diffraction (XRPD) pattern which gives calculated lattice spacings at 10.29, 10.76, 11.94, 14.33, 14.61, 14.93, 16.02, 16.80, 17.47, 17.92, 19.13, 19.55, 19.84, 20.33, 21.16, 21.36, 23.33, 23.96, 24.44, 24.67, 25.51, 26.12, 27.13, 27.77, 28.06, 28.35, 29.23, 29.46, 30.06, 30.35, 31.27, 32.35, 32.66, 33.08, 33.77, 34.49, 35.18, 36.42, 37.34, 38.39 and 39.51°.

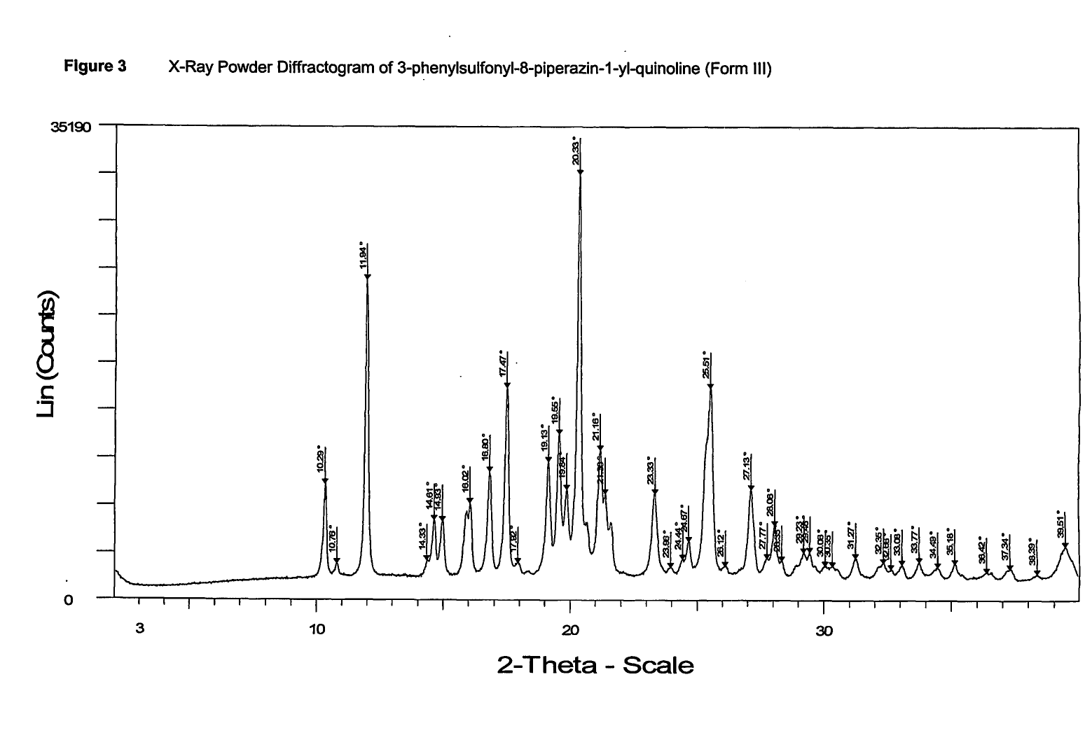

5. A polymorph according to claim 1 which provides an X-ray powder diffraction (XRPD) pattern substantially in accordance with FIG. 3.

6. A polymorph according to claim 1, in isolated form.

7. A polymorph according to claim 1, in pure form.

8. A polymorph according to claim 1, in crystalline form.

9. A pharmaceutical composition which comprises a polymorph according to claim 1 and a pharmaceutically acceptable carrier or excipient.

10. A polymorph according to claim 1 for use in therapy.

11-12. (canceled)

13. A pharmaceutical composition comprising a polymorph according to claim 1 for use in the treatment of depression, anxiety, Alzheimers disease, age related cognitive decline, ADHD, obesity, mild cognitive impairment, schizophrenia, cognitive deficits in schizophrenia and stroke.

14. A method of treating depression, anxiety, Alzheimers disease, age related cognitive decline, ADHD, obesity, mild cognitive impairment, schizophrenia, cognitive deficits in schizophrenia and stroke which comprises administering a safe and therapeutically effective amount to a patient in need thereof of a polymorph according to claim 1.

15. A method of promoting neuronal growth within the central nervous system of a mammal which comprises the step of administering a polymorph according to claim 1.

16. (canceled)

17. A pharmaceutical composition comprising a polymorph according to claim 1 for use in promoting neuronal growth within the central nervous system of a mammal.
